(19) United States
(12) Patent Application Publication
(10) Pub. No.: US 2015/0238686 A1
(43) Pub. Date: Aug. 27, 2015

(54) Disposable Multiuse Syringe Holder

(71) Applicant: John Cyrus Mohyi, La Jolla, CA (US)

(72) Inventor: John Cyrus Mohyi, La Jolla, CA (US)

(21) Appl. No.: 14/192,589

(22) Filed: Feb. 27, 2014

(51) Int. Cl. A61M 5/00

(52) U.S. Cl. CPC A61M 5/008

(57) ABSTRACT

The present invention is a disposable multiuse syringe holder and organizer which secures the syringes through the use of elastic pressure in its syringe holes or slots. It is a minimalist design which eliminates excess material both reducing material and fabrication costs. It is composed of a slightly elastic material such as foam board, cardboard, rubber, cork, corrugated plastic or other material with similar elastic properties. The device is intended to have a minimal footprint and the disposable nature of the device lends itself to be written on to indicate information about the syringes being held. An additional advantage in a standard rectangular configuration is that syringes held on the device can be stacked conveniently in a consecutively reversing formation.

The present invention relates to a low cost, multiuse, disposable syringe holder and organizer.

In its most basic form, a syringe holder is a device with numerous holes, openings, or slots where a syringe or syringes may be inserted for the purpose of organization and storage.

Current syringe holders are generally composed of plastic, metal, wood, or a comparable rigid material. Their designs tend to have a larger than necessary footprint and corresponding greater weight which is less economical in terms of both cost and storage. Further, their non-disposable nature does not make them ideal for being written on to add value in terms of important notes or dates.

In contrast, the present invention is a minimalist design which leverages the suitably elastic nature of the material to achieve its purpose of holding the syringe via elastic pressure. It achieves its purpose when the ratio between the syringe diameter and the hole diameter for holding the syringe are within the right tolerance range for adequacy holding onto the syringe cap without unnecessarily increasing the difficulty of placing the syringe in the hole or removing it. Further, the degree of elastic pressure placed on the syringe from the material, when placed in the hole, must not exceed the force required to separate the syringe cap from the body of the syringe.

### SUMMARY

The present invention comprises a multiuse disposable syringe holder and organizer. The device in one embodiment is of a rectangular dimension with multiple circular holes which act as syringe holders when the elastic pressure of the material interacts with the syringe surface. It is comprised of a material which is flexible enough to expand to accommodate a slightly larger gauge needle cap in its circular holes providing sufficient friction on the cap of the syringe to hold it in place, but less friction than the force required to separate the cap from the syringe itself. The optimal gauge of the hole relative to the syringe varies slightly depending on material properties and the degree of surface area interacting between the syringe holder and syringe cap or other surface of the syringe. The holes are arranged in a row or grid formation and are separated by a minimum of the distance required to provide adequate clearance between syringes based upon the horizontal diameter of the syringe being used. Additional material may be used to enhance: material rigidity, space for written notes, surface area for handling, logo space, or for other additional functionality.

### BRIEF DESCRIPTION OF THE SEVERAL VIEWS OF THE DRAWINGS

The foregoing summary, as well as the detailed description of the preferred embodiments of the present invention, will be better understood when read in conjunction with the appended drawings. For the purpose of illustrating the invention, there is shown in the drawings, which are diagrammatic, embodiments that are presently preferred. It should be understood, however, that the present invention is not limited to the precise arrangements and instrumentalities shown. In the drawings:

FIG. 1 is an angled perspective view of the invention with syringes.

FIG. 2 is an angled perspective view of the invention without syringes.

FIG. 3 is a perspective top angle view of the invention with syringes.

FIG. 4 is a perspective top angle view of the invention without syringes.

FIG. 5 is a perspective “long side” angle view of the invention with syringes.

FIG. 6 is a perspective “long side” angle view of the invention without syringes.

FIG. 7 is a perspective “short side” angle view of the invention with syringes.

FIG. 8 is a perspective “short side” angle view of the invention without syringes.

FIG. 9 depicts an example of a syringe that may be used in the device.

FIG. 10 is a perspective view of full syringe holders stacked in a consecutively reversing formation.

### DETAILED DESCRIPTION OF THE INVENTION

Certain terminology is used in the following description for convenience only and is not limiting. The word “Hole(s)” is used to describe the slots or holding area for the syringes and “Elastic Pressure” refers to the force exerted on the surface of the syringe from the material attempting to assume its original configuration before accommodating for the syringe.

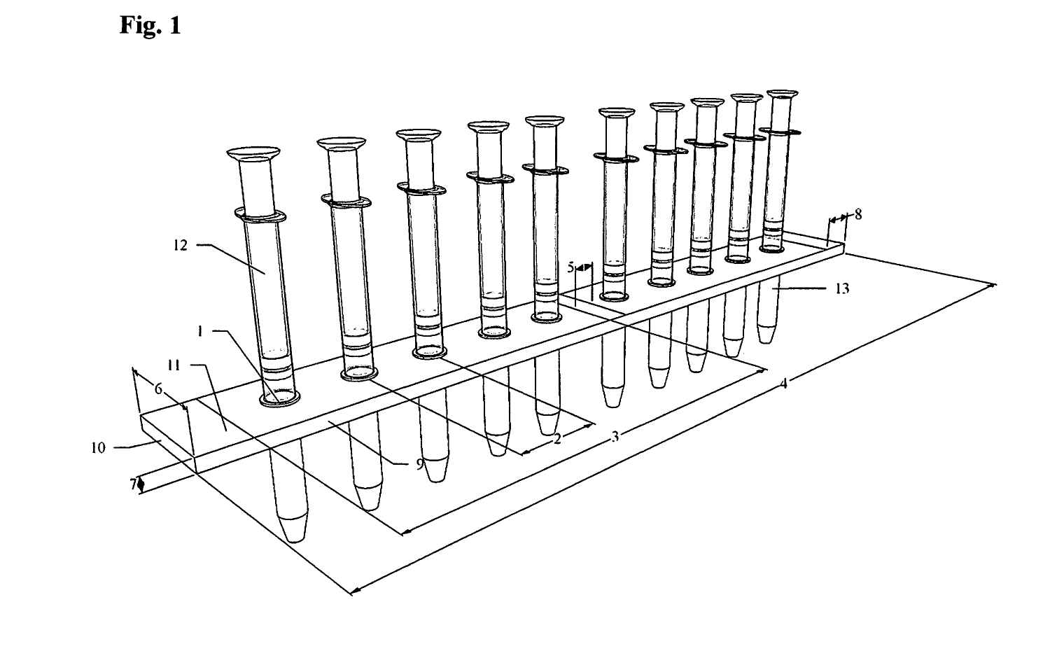

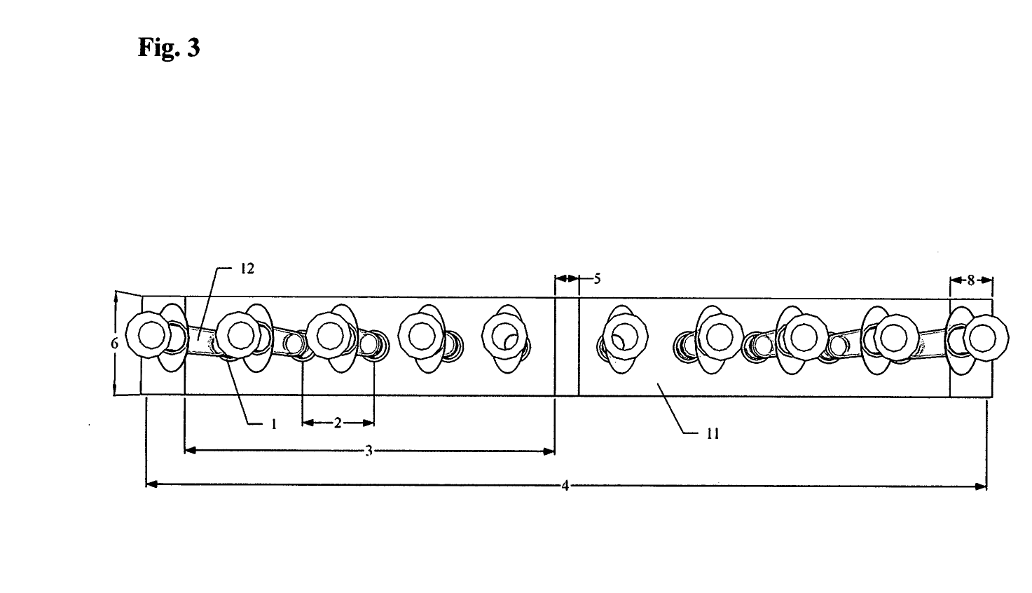

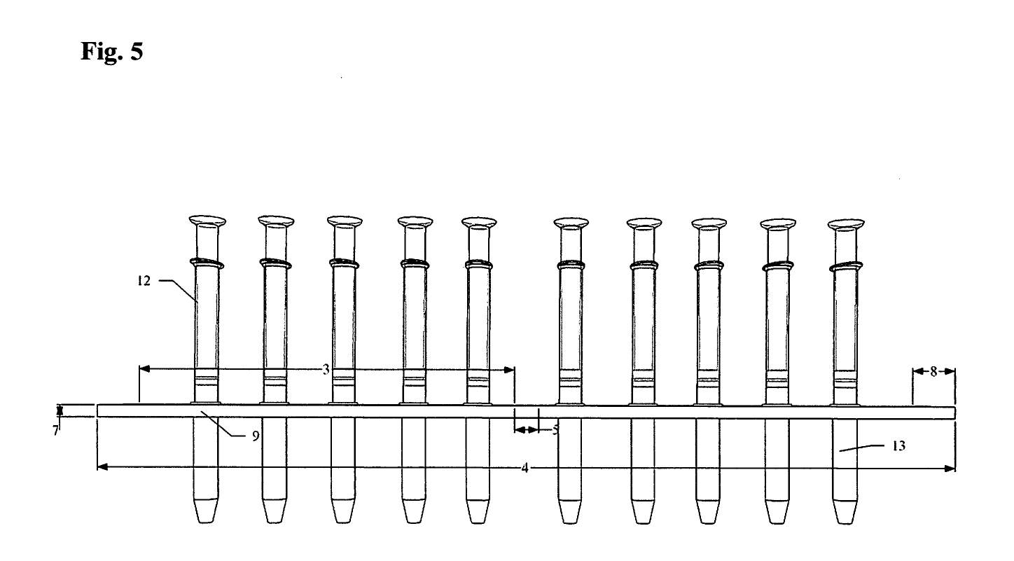

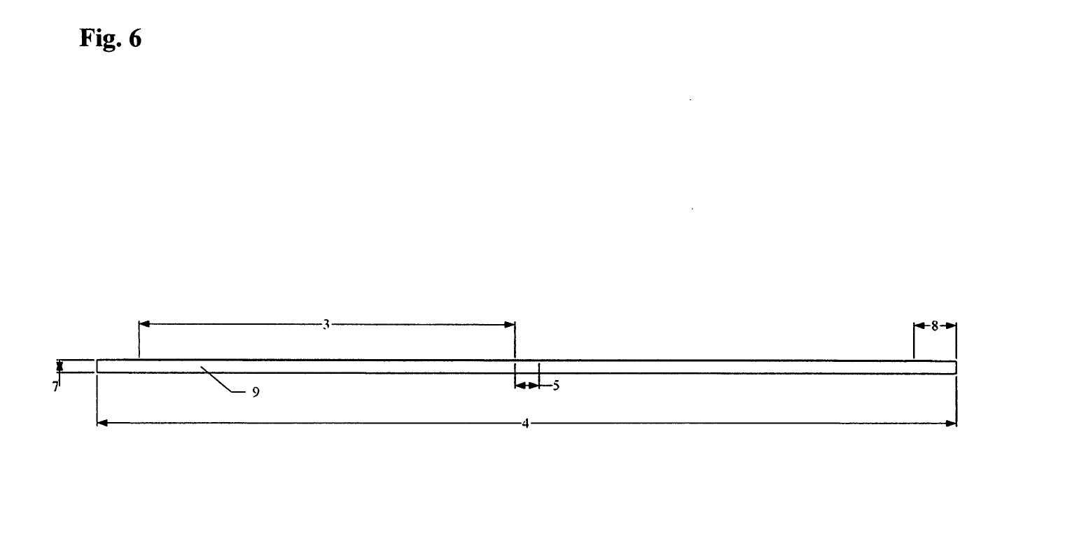

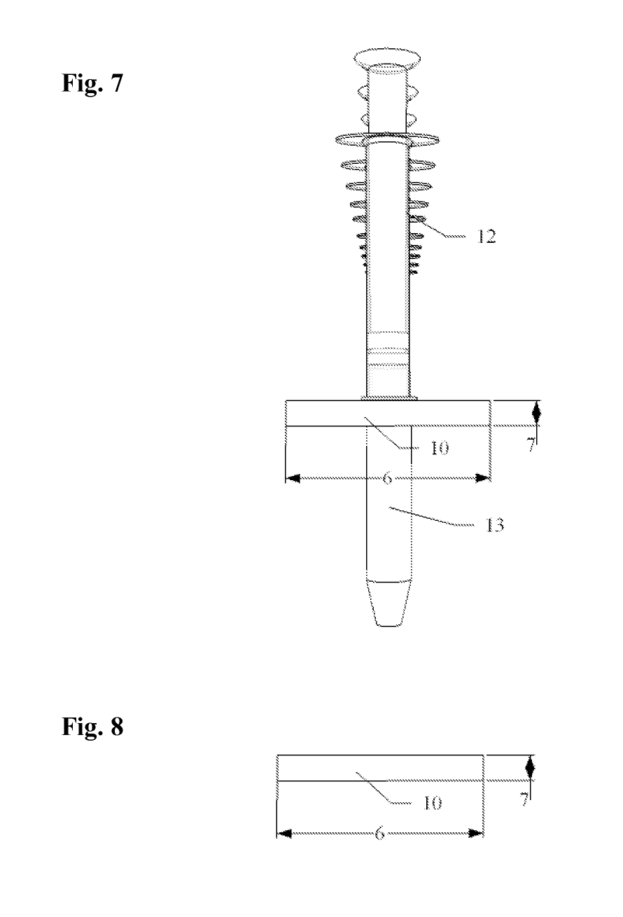

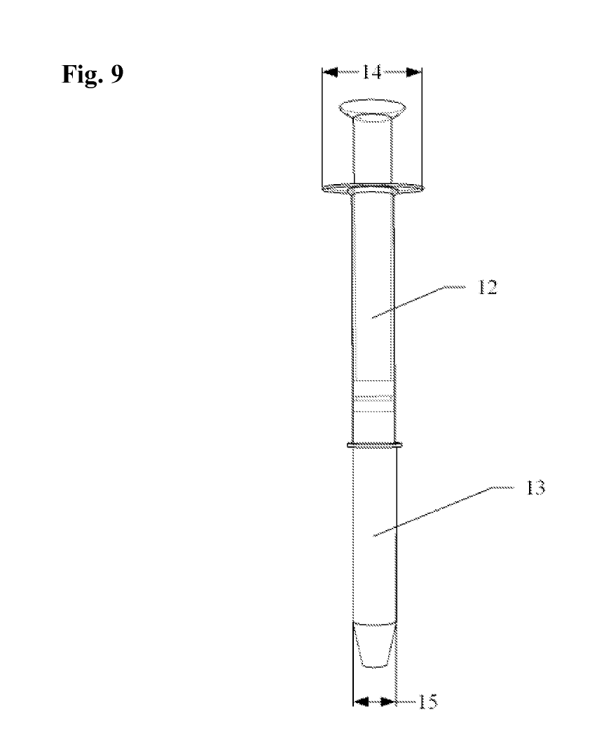

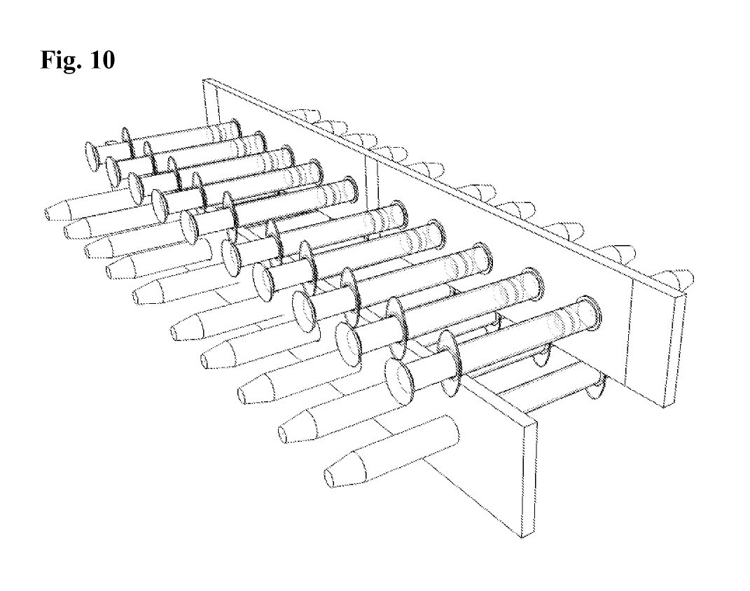

Referring now to the drawings and diagrams in detail, wherein like numbers are used to indicate like elements throughout, there is shown in FIGS. 1-8 the device of the invention, i.e. a low cost multiuse syringe holder. FIG. 9 depicts an example of a syringe that may be used in the device and FIG. 10 depicts a possible formation with two or more syringe holders in use.

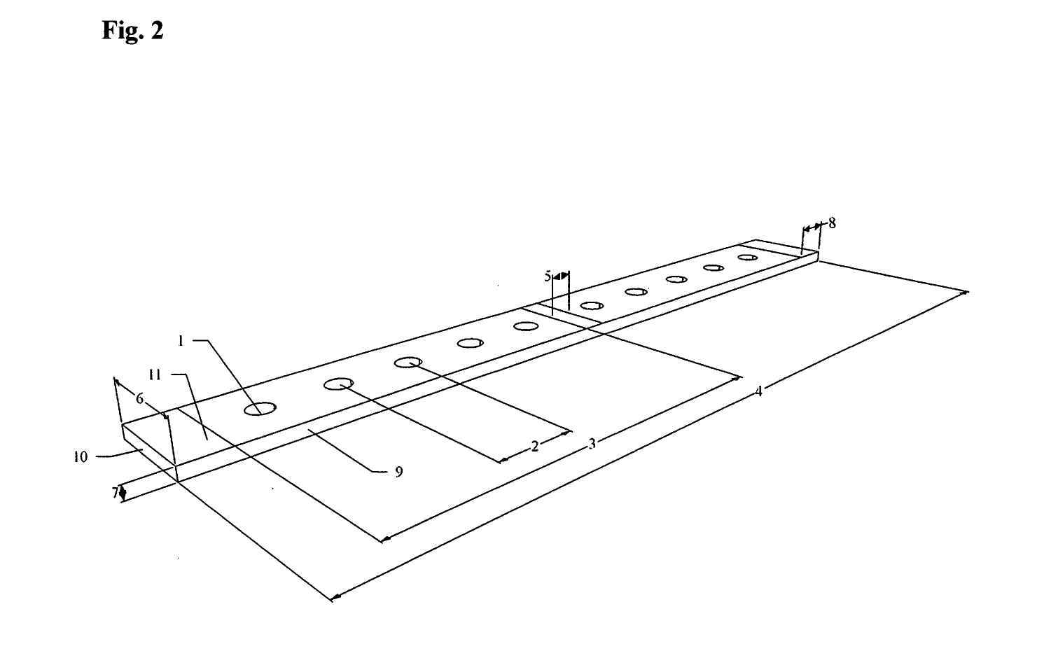

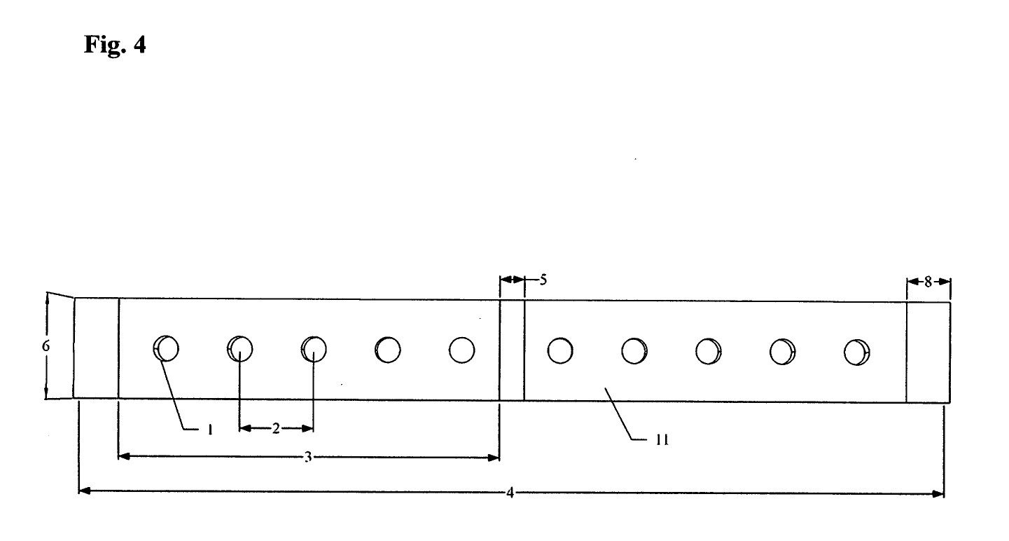

Referring to FIGS. 1, 2, 3, 4, 5, 6, 7, and 8 the syringe holder assembly basically comprises a disposable multiuse syringe holder. In one embodiment of the invention designed for use with 0.3CC BD insulin syringes, a foam core board or cardboard material with a length 4 as depicted in FIGS. 1-6, width 6 as depicted in FIGS. 1-4 7 and 8, and thickness 7 as depicted in FIGS. 1, 2, 5-8 has ten holes 1 as depicted best in FIGS. 2 and 4 divided into sets 3 as depicted in FIGS. 1-6 of five for ease of counting. Each hole 1 has a diameter approximately 1 to 50 percent less than the diameter of the syringe cap 13 as depicted in FIGS. 1, 5, 7, and 9, depending on the material and desired elastic pressure, and each hole 1 is separated by the distance 2 as depicted in FIGS. 1-4 required to provide clearance between each syringe or approximately 1.5 cm from hole center point to hole center point. Each set 3 of five holes is separated by an additional length 5 as depicted in FIGS. 1-6 which measures approximately 0.5 cm or any length suitable for enabling a viewer to easily distinguish between sets 3 of holes. The distance between the holes 1 and the edge of the long side 4 of the device corresponds to the maximum horizontal diameter of the syringe 14 as shown in FIG. 8. Additional material to length 4, width 6, or thickness 7 may be added to aid in the structural rigidity of the device, to increase writing area, and to enhance handling of the device. The distance between the holes 1 and the short edge 6 of the device corresponds to the maximum diameter of the syringe 14 as shown in FIG. 8 plus an extra length of material 8 as depicted in FIGS. 1-6 of approximately 1 cm to each end of long side 4 to enhance handling of the device. FIG. 10 depicts full syringe holders stacked in a consecutively reversing formation for ease of storage.

### Index of Reference Numbers 1-14

Reference Number 1: Indicates the holes, slots, or openings the syringe fits into.
Reference Number 2: Indicates the distance from center-point to center-point of adjacent holes in the same set.
Reference Number 3: Indicates a set of five holes.
Reference Number 4: Indicates the long edge and the entire length of the device.
Reference Number 5: Indicates a visual buffer between sets of holes.
Reference Number 6: Indicates the short edge and the width of the device.
Reference Number 7: Indicates the thickness of the device
Reference Number 8: Indicates additional material at the ends of the long edge of the device for enhancing: handling, writing area, and structural support of the device.
Reference Number 9: Indicates the long face side of the device.
Reference Number 10: Indicates the short face side of the device.
Reference Number 11: Indicates the top face of the device.
Reference Number 12: Indicates the body of the syringe.
Reference Number 13: Indicates the cap of the syringe.
Reference Number 14: Indicates the maximum horizontal width of the syringe.
Reference Number 15: Indicates the diameter of the syringe cap.

### Structural Dimensions of Syringe Holder

The structural dimensions of the syringe holder are optimized based upon the dimensions of the syringe to be used. The structural dimensions below indicate how they can be derived for a particular syringe, but they should be interpreted as a general guide and may be modified while still maintaining the spirit of the invention. The measurements listed to the far right indicate a possible example, and are not intended to narrow the scope of the claim to said measurements.
[Hole 1]=[Cap/Surface Diameter 15]−[1-50% Cap/Surface Diameter]=˜<5 mm
[Width 6]=[Max Horizontal Width of Syringe 14]=˜1.5 cm
[Set 3]=[Max Horizontal Width of Syringe 14]×5=˜7.5 cm
[End Material 8]=˜1 cm
[Buffer 5]=˜0.5 cm
[Length 4]=[End Length 8]+[Set 3]+[Buffer 5]+[Set 3]+[End Length 8]=˜17.5 cm

What is claimed is:

1. A multiuse disposable syringe holder comprising
a. A semi flexible material such as foam core board, cardboard, rubber, cork, corrugated plastic, or comparable material in sheet form with a thickness which may range from 1 mm to 1 cm;
b. A plurality of holes or slots for holding syringes arranged in a row or grid like formation for the purpose of holding syringes via the elastic pressure of the material interacting with the syringe cap or other surface of a syringe;
c. A elastic pressure of the holes or slots high enough to safely secure the syringe, but less than the force required to separate the syringe cap from the body of the syringe upon removal for the syringe from the hole while holding the body of the syringe;

2. The syringe holder of claim 1 which is durable enough to endure multiple uses, but affordable enough because of its simplicity and low material cost to be considered disposable.

3. A syringe holder, which due to its disposable nature, can be written on or easily marked for the purpose of indicating information about the contents of a syringe or syringes.

4. The syringe holder of claim 1 which when filled with syringes can be horizontally stacked upon other syringe holders filled with syringes in a consecutively reversing formation.
